(19) United States
(12) Patent Application Publication
(10) Pub. No.: US 2024/0157040 A1
(43) Pub. Date: May 16, 2024

(54) ENEMA ASSIST DEVICE

(71) Applicant: The Children's Hospital of Philadelphia, Philadelphia, PA (US)

(72) Inventors: Joy Kerr, Philadelphia, PA (US); Michelle Hsia, Philadelphia, PA (US); Jason Van Batavia, Philadelphia, PA (US)

(73) Assignee: The Children's Hospital of Philadelphia, Philadelphia, PA (US)

(21) Appl. No.: 18/282,653

(22) PCT Filed: Mar. 22, 2022
(86) PCT No.: PCT/US2022/021304

§ 371 Date: Sep. 18, 2023

Related U.S. Application Data
(60) Provisional application No. 63/164,811, filed on Mar. 23, 2021.

(51) Int. Cl. A61M 3/02

(52) U.S. Cl. CPC A61M 3/0225; A61M 3/0279

(57) ABSTRACT

An enema assist device is provided. The device includes a support and the support is configured to be placed on a toilet bowl such that the support front is oriented towards the front of the toilet bowl. The support is configured to support a fluid delivery tip such that the fluid delivery tip is disposed on the top surface of the support and protrudes at least partially beyond a plane defined by a top of the toilet seat.

### CROSS-REFERENCE TO RELATED APPLICATIONS

The present application claims priority to U.S. Provisional Patent Application No. 63/164,811, filed Mar. 23, 2021, entitled “ENEMA ASSIST DEVICE” the contents of which are incorporated herein by reference in their entirety.

### FIELD

The subject matter disclosed herein relates to devices and methods for providing enemas to a patient.

### BACKGROUND

Spina bifida, which literally means “cleft spine”, is characterized by the incomplete development of the brain, spinal cord, and/or the meninges (the protective covering around the brain and spinal cord). Patients with spina bifida have damaged nerves that affect the bowel and the anal sphincter muscle. In particular, both the bowel and the sphincter have low muscle tone, leading to the combined problems of constipation and bowel movements at unexpected times, i.e., bowel incontinence. The constipation is because the bowel cannot effectively evacuate stool and the bowel incontinence is because the anal sphincter is relatively weak and cannot hold the stool. The end result is called neurogenic bowel and is common in spina bifida and other neurological conditions, such as but not limited to spinal cord injury, multiple sclerosis, stroke, transverse myelitis, and others. Therefore, in the spina bifida population and other patients with neurogenic bowel, daily high-volume enemas are needed to fully the evacuate the bowel and prevent both of these problems.

Cone enemas, in which the enema is delivered to the bowel using a tapered, or cone-shaped tip for the enema insertion is used because, if held firmly to the weak anal sphincter, it can provide a water tight seal. However, the current cone enema systems do not allow for a reproducibly water tight seal between the cone and the bowel, since skill and practice are needed for effective self-administration of these devices. Delivering the enemas can be challenging, especially for pediatric patients and their care givers. The delivery of the enema and evacuation of the bowel may take from 45 minutes to an hour. Naturally, such skill (not to mention patience) is more difficult for pediatric patients. The result is that leakage occurs during administration of the enema fluid, which results in inadequate administration of the enema fluid, as well as waste of the fluid, and low adherence to the bowel evacuation regimen.

The current solution to this problem is that the enemas are administered with assistance from a caregiver. Thus, the patients and their families experience high stress related to need for these enemas. The patients are also frustrated due to lack of independence.

Other efforts to solve this problem are summarized in the following documents:

P. Midrio, et al., Peristeen® transanal irrigation in paediatric patients with anorectal malformations and spinal cord lesions: a multicentre Italian study, Colorectal Dis. 2016 January; 18(1):86-93; doi: 10.1111/codi.13101 discloses a system involving involves a semi-rigid catheter with a balloon to hold the device in place. The problem with this system is that because spina bifida patients have poor anal sphincter muscle tone, the balloon is often expelled by the patient before the enema is complete, therefore a caregiver must hold the catheter in. In addition, the device is not disclosed to be suitable to attach to a toilet.

US Patent Application Publ. No. US 2004/0148689 A1 discloses a bidet attachment for a toilet. It is not disclosed to be suitable for providing a water tight seal to provide an enema to a person having poor anal sphincter muscle tone.

US Patent Application Publ. No. US 2005/0223482 A1 discloses an irrigation system that can be used while sitting on the toilet. The device is not disclosed to be suitable for providing a water tight seal to provide an enema to a person having poor anal sphincter muscle tone.

US Patent Application Publ. No. US 2019/0224412 A1 discloses a trans-anal irrigation device. It is not disclosed to be suitable to attach to a toilet.

U.S. Pat. No. 4,205,402 A discloses bidet attachment for a toilet. It is not disclosed to be suitable for providing a water tight seal to provide an enema to a person having poor anal sphincter muscle tone.

U.S. Pat. No. RE38,690 E discloses a toilet attachment for an enema, douche or colonic irrigation. The device has an irrigation tip that further has a delimiter to prevent the tip from being inserted too far into a body cavity. The tip is not disclosed to be suitable for providing a water tight seal in a person having poor anal sphincter muscle tone.

U.S. Pat. No. 6,964,653 B2 discloses a bacteria inhibiting hygienic device for vaginal/colon cleansing. The device is not disclosed to be attachable to a toilet, nor is it disclosed to be suitable for providing a water tight seal in a person having poor anal sphincter muscle tone.

Accordingly, there remains a need for a device that effectively and reproducibly holds the cone enema systems in place, without need for assistance.

### SUMMARY

Four exemplary embodiments of an enema assist device are described herein. The enema assist device is intended to help patients having a weak anal sphincter muscle become independent with respect to enema administration. In particular, all of the embodiments are intended to hold a fluid delivery tip for an enema system in place above a toilet so that a patient in need of such an enema assist device can adjust the position of the fluid delivery tip as necessary, and then simply sit on the fluid delivery tip to create a water tight seal between the fluid delivery tip and the weak anal sphincter muscle. Accordingly, all of the embodiments as disclosed herein are capable of holding a fluid delivery tip for an enema system in a fixed position when the fluid delivery tip is sat on.

A “potty training” embodiment works with a cone irrigator device used in the spina bifida and neurogenic bowel population to deliver high volume enemas for daily bowel management. A potty-training toilet seat is adapted with a support to hold the fluid delivery tip that allows a patient, in particular a child, to sit on the device to create a water tight seal thereby eliminating the need for parents to reach over and behind the child to hold the fluid delivery tip in place. In another embodiment, this system has also been adapted for adults—no training seat.

Another embodiment is for an older child/adolescent or adult that enable the fluid delivery tip to be attached to the end of a curved metal bar that enables the patient to place the tip in the anatomically correct position and push down with their body weight to create a water tight seal. In all of these embodiments, the common element is that the fluid delivery tip is cone shaped and that the tip is supported over the toilet bowl, such that the body weight of the patient is used to affect a water tight seal of the conical fluid delivery tip to the weak anal sphincter.

In the fourth embodiment, the fluid delivery tip may be attached on the end of a curved metal bar. In this embodiment as in the other three, the end of the curved metal bar may be equipped with a platform and/or wedge that is capable of angling the fluid delivery tip so as to alter the delivery angle to best seal the tip to a user's anal sphincter. In this embodiment, the curved metal bar may be simply cantilevered on the edge of the toilet bowl and the user only need apply a downward force on a handle portion of the curved metal bar to achieve suitable resistance to their body weight to hold the tip in the sealed position.

It should be understood that the wedges described in this fourth embodiment may be used with any of the embodiments disclosed herein.

An enema assist device is provided. The enema assist device includes a support. The support has a front, a back, a bottom surface, and a top surface, wherein the support is configured to be placed on a toilet bowl such that the support front is oriented towards the front of the toilet bowl. The support is further configured to support a fluid delivery tip such that the fluid delivery tip is disposed on the top surface of the support and protrudes at least partially beyond a plane defined by a top of the toilet seat.

According to another embodiment, the fluid delivery tip may include a conic section that has a top with an upper diameter and a bottom with a lower diameter. The upper diameter is smaller than the lower diameter, and the bottom of the conic section is disposed on the top surface of the support. In an embodiment, the top surface of the support may slope at an angle α, relative to the plane defined by the top of the toilet seat, thereby angling the fluid delivery tip at the angle α.

According to a further embodiment, the support may further include at least one angled wedge section on the top surface thereof. The angled wedge section is interposed between the fluid delivery tip and the support, the wedge having an angle α, relative to the plane defined by the top of the toilet seat, thereby imparting the angle α to the top of the support and thus angling the fluid delivery tip at the angle α. The angled wedge may further have a rotational angle β relative to an axis along the length of the support. The top surface of the support may slope downward from back to front at an acute angle α, thereby positioning the fluid delivery tip at an angle toward the toilet front. The support may include a platform, the platform having a first side and an opposing second side, such that the first side and the second side of the platform are placed under opposing sides of the toilet seat, and the support front is oriented towards the toilet front.

In an embodiment the enema assist device may include a removable toilet seat configured to be placed on top of a fixed toilet seat on a toilet bowl.

According to another embodiment, the support may include a rigid cantilever. The rigid cantilever may also include an adjustable attachment system, a first end and a second end, such that the first end is attached to a toilet bowl under the toilet seat with the adjustable attachment system and the second end is attachable to the fluid delivery tip. The adjustable attachment system may include at least one clamp configured to attach to the toilet bowl, and an adjustment clip fixedly attached to the at least one clamp. The adjustment clip may include a lower receptacle configured to hold the first end and an upper arm pivotally attached to the lower receptacle and configured to pivot between an open position and a closed position. The adjustment clip also includes a spring attached to the lower receptacle and to the upper arm. In operation, when the upper arm is in the open position, the first end can be placed in the lower receptacle and be moved from front to back relative to the toilet bowl, and when the upper arm is in the closed position, the spring urges the upper arm against the first end, such that the first end is attached to the toilet bowl. According to an embodiment, the rigid cantilever may be a curved bar. According to an embodiment, the second end may further include at least one angled wedge section on the top surface thereof and interposed between the fluid delivery tip and the second end. The wedge has an angle α, relative to the plane defined by the top of the toilet seat. When the wedge is place, it serves to impart the angle α to the top of the top surface of the second end and thus angling the fluid delivery tip at the angle α.

According to another embodiment, the support is a rigid bar. In this embodiment, the rigid bar has a hand grip region located at the front of the rigid bar, an attachment region located on the top surface of the back of the rigid bar, and a pivot region located on the bottom surface and in between the front and the back of the rigid bar. The rigid bar is configured such that the fluid delivery tip is attachable to the attachment region. In use, the pivot region contacts the toilet bowl such that the hand grip region projects beyond an edge of the toilet bowl, the fluid delivery tip is positioned over a center region of the toilet bowl, and a user of the enema assist device grips the hand grip region and urges the hand grip region downward and thereby pivots the fluid delivery tip upward and urges the fluid delivery tip into the body orifice of the user. The fluid delivery tip attachment region may further include at least one angled wedge section on the top surface thereof and interposed between the fluid delivery tip and the support. The wedge section has an angle α, relative to the plane defined by the top of the toilet seat, thereby imparting the angle α to the top of the support and thus angling the fluid delivery tip at the angle α. The wedge section may also have a rotational angle β, measured relative to an axis along the length of the rigid bar. The rigid bar may also be curved.

### BRIEF DESCRIPTION OF THE DRAWINGS

FIG. 1 shows a cone enema system;

FIG. 2 shows a view of an exemplary embodiment of a fluid delivery tip according to the present disclosure;

FIG. 3 shows a view of an exemplary first embodiment of the present disclosure;

FIG. 4 shows a second view of the exemplary first embodiment of the present disclosure;

FIG. 5 shows a view of an exemplary second embodiment of the present disclosure;

FIG. 6 shows a second view of the exemplary second embodiment of the present disclosure;

FIG. 7 shows a third view of the exemplary second embodiment of the present disclosure;

FIG. 8 shows a fourth view of the exemplary second embodiment of the present disclosure;

FIG. 9 shows a fifth view of the exemplary second embodiment of the present disclosure;

FIG. 10 shows a sixth view of the exemplary second embodiment of the present disclosure;

FIG. 11 shows a view of an exemplary third embodiment of the present disclosure;

FIG. 12 shows a second view of the exemplary third embodiment of the present disclosure;

FIG. 13 shows a third view of the exemplary third embodiment of the present disclosure;

FIGS. 14A, 14B and 14C show a perspective view, a side view, and a top view, respectively, of an exemplary fourth embodiment of the present disclosure;

FIG. 15 shows an exploded view of the exemplary fourth embodiment of the present disclosure; and

FIGS. 16A-G show portions of the exemplary fourth embodiment of the present disclosure.

### DETAILED DESCRIPTION

Although the present disclosure makes reference to specific embodiments, the present disclosure is not intended to be limited to the details shown. Rather, various modifications may be made in the details within the scope and range of equivalents of the claims and without departing from the present disclosure.

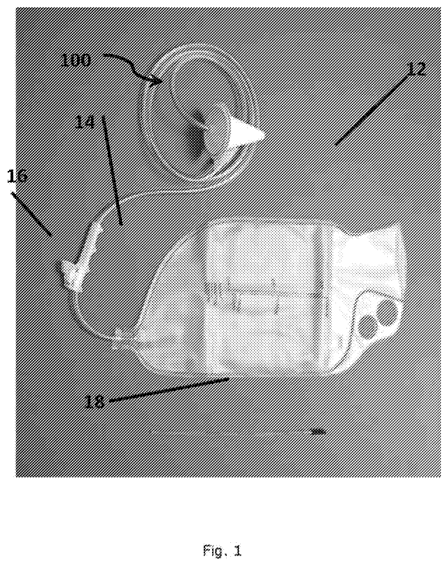

Referring to FIG. 1, an enema system 100 is shown. The enema system may comprise a fluid container 12, which is in fluid communication with a fluid delivery tip 14 via a tubing 16. As shown in this exemplary embodiment, a flow control device 18 is associated with the tubing 16. The flow control device 18 is constructed and arranged so that a user of the enema system can control the flow rate of fluid from the fluid contain 12 through the tubing to the fluid delivery tip 14. In an embodiment, the enema system 100 may be “gravity fed”, meaning that hydraulic pressure to feed the enema fluid from the fluid container 12 through the tubing 16, the flow control device 18, the fluid delivery tip 14 and ultimately into a body orifice of a patient in need of an enema is a result of holding the fluid container 12 above the fluid delivery tip 14.

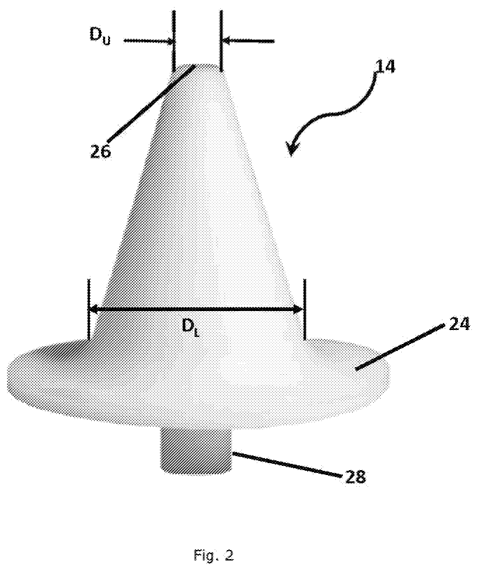

FIG. 2 shows an embodiment of the fluid delivery tip 14. As shown in FIG. 2, the fluid delivery tip 14 is generally conical in shape and may be considered to be a conic section, having a top 20 with an upper outer diameter D_{U} and a bottom 22 with a lower outer diameter D_{L}, where D_{U}<D_{L}. As shown in the embodiment of FIG. 2, the bottom 22 may optionally comprise a shoulder section 24. The fluid delivery tip comprises a through hole 26 and also a tubing adaptor 28. The tubing adaptor is constructed and arranged to connect to the tubing 16 to provide a fluid communication between the fluid container 12 and the fluid delivery tip 14.

The top 20 of the fluid delivery tip 14 is constructed and arranged to be partially insertable into a body orifice of a user, i.e., an anal sphincter of a patient using the enema system 100. The bottom 22 of the fluid delivery tip 14 is constructed and arranged to provide a tight seal to the anal sphincter of a patient using the enema system 100, even if, and especially if, the anal sphincter has poor muscle tone. The upper outer diameter D_{U} may be from 0.3 cm to 1 cm and the lower outer diameter D_{L} may be from 1.5 cm to 3 cm. The fluid delivery tip 14 may have a bilayer construction in which an outer layer is comprised of a relatively soft material, such as silicone, plasticized polyvinylchloride (PVC) and the like, while an inner layer is a relatively rigid material, such as unplasticized PVC, polypropylene, high density polyethylene, polystyrene, polymerized acrylics, and the like. In this way, the fluid delivery tip 14 may conform to the anal sphincter of a patient during use of the enema, but the rigid interior provides a water tight seal to a weak anal sphincter when the fluid delivery tip 14 is in use.

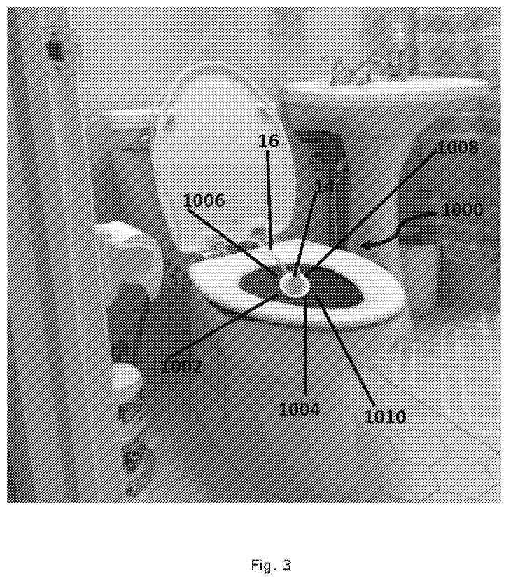

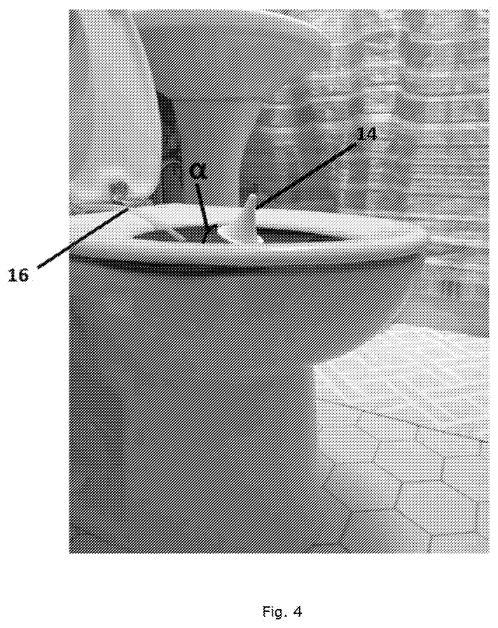

FIGS. 3 and 4 show views of an exemplary first embodiment enema assist device 1000 in place on a toilet. As seen in FIG. 3, the first embodiment enema assist device 1000 comprises a support 1002 for fluid delivery tip 14. The support 1002 has a front 1004, a back 1006, a bottom surface 1008 and a top surface 1010. The support 1002 is placed under the toilet seat and is configured so that the fluid delivery tip 14 can be disposed on the top surface 1010 of the support 1002. The support front 1004 faces the front of the toilet. As seen more clearly in FIG. 4, the support 1002 is configured to support the fluid delivery tip 14 such that when the fluid delivery tip 14 is disposed on the top surface of the support, the fluid delivery tip 14 protrudes at least partially beyond a plane defined by a top of the toilet seat. The tubing 16 for the enema system is arranged to allow fluid communication between the fluid delivery tip 14 and the fluid container 12 (not shown in FIGS. 3 and 4). Further, as can be seen in both FIG. 3 and FIG. 4 (most clearly in FIG. 4) the top surface 1010 of the support 1002 may slopes downward from back 1006 to front 1004 at an acute angle α, thereby positioning the fluid delivery tip 14 at an angle toward the toilet front. The support 1002 may has two opposing sides 1014 and 1016 that are placed under the toilet seat.

In use, a patient in need of the enema assist device 1000 as disclosed herein, i.e. a person having weak muscle tone in the anal sphincter, would place the enema assist device 1000 under a toilet seat. The enema assist device 1000 may be moved backwards and forwards relative to the toilet seat, depending on the size of the toilet seat and the size of the patient. The patient would then sit on the toilet seat such that the fluid delivery tip 14 would partially enter their colon, since the fluid delivery tip protrudes beyond the plane of the toilet seat. In addition, the support 1002 is sufficiently rigid that it does not appreciably deflect due to the person's weight. Because the fluid delivery tip 14 is tapered, i.e., has a conical shape, and is soft on the outside, but comprises a rigid interior, a water tight seal is formed between the fluid delivery tip 14 and an anal sphincter having weak muscle tone. The enema assist device 1000 thus eliminates the need for a caregiver to reach over and behind the patient to hold the fluid delivery tip in place.

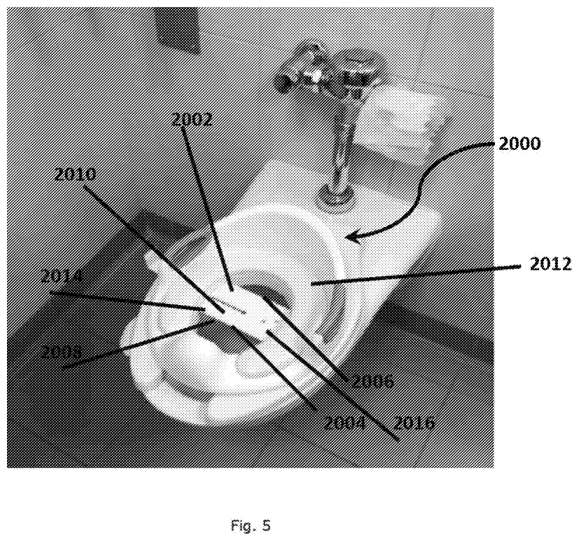

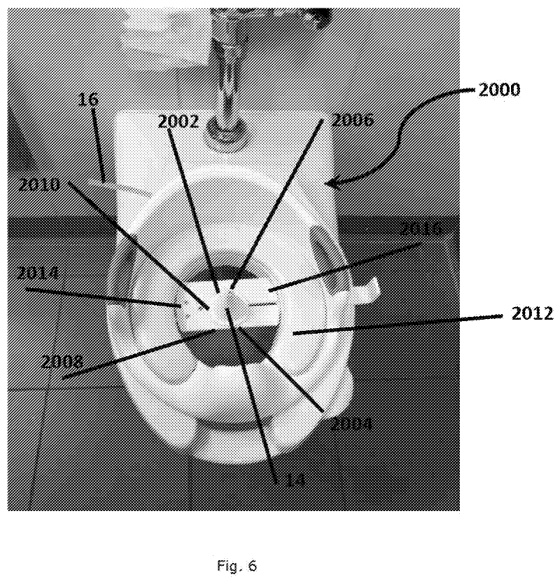

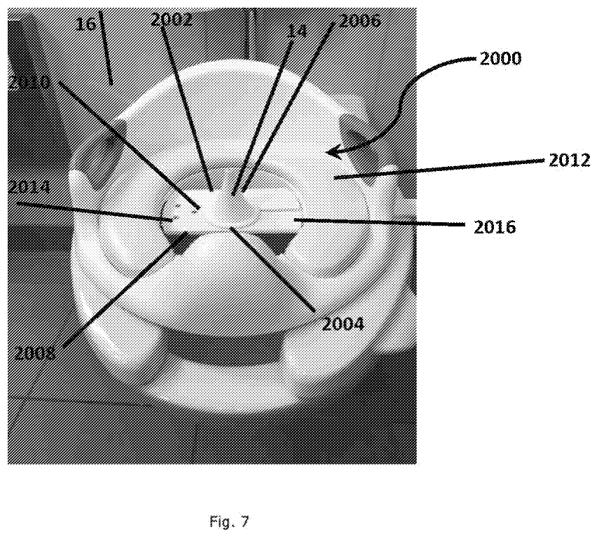

FIGS. 5-7 show views of an exemplary second embodiment enema assist device 2000 in place on a toilet. As seen in FIG. 5, the second embodiment enema assist device 2000 comprises a support 2002 for fluid delivery tip 14 (not shown in FIG. 5). The support 2002 has a front 2004, a back 2006, a bottom surface 2008 and a top surface 2010. The support 2002 is placed under a removable toilet seat 2012 and the fluid delivery tip 14 is disposed on the top surface 2010 of the support 2002. The support front 2004 faces the front of the toilet. As can be seen more clearly in FIG. 7, the support 2002 is configured to support the fluid delivery tip 14 such that when the fluid delivery tip 14 is disposed on the top surface of the support, the fluid delivery tip 14 protrudes at least partially beyond a plane defined by a top of the toilet seat. As can be seen in both FIGS. 5-7, the tubing 16 for the enema system is arranged to allow fluid communication between the fluid delivery tip 14 and the fluid container 12 (not shown in FIGS. 5-7). The support 2002 may have two opposing sides 2014 and 2016 that are placed under the removable toilet seat 2012.

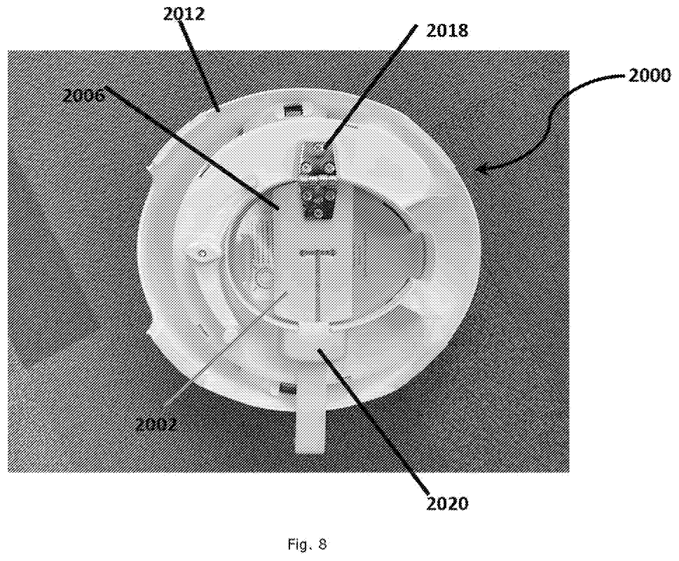

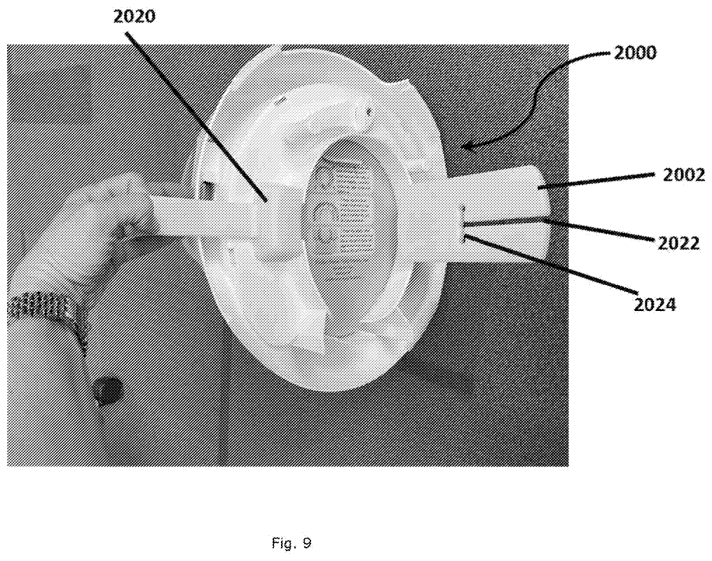

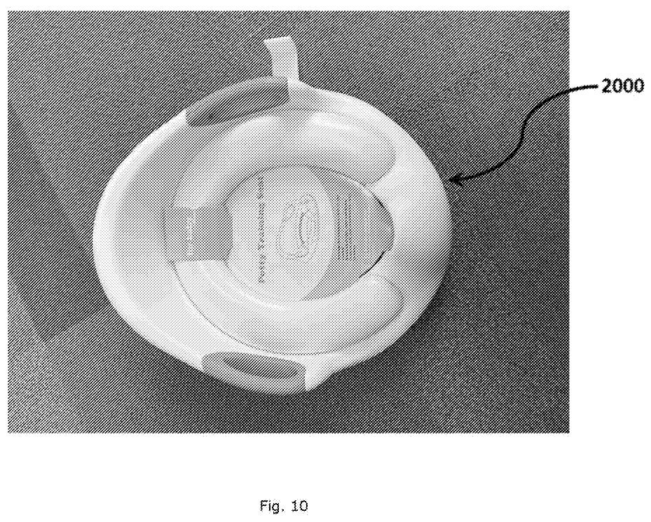

FIGS. 8-10 show bottom, bottom perspective, top views, respectively, of the second embodiment enema assist device 2000. The bottom view of the of the enema assist device 2000 shows the bottom surface 2008 of the support 2002 and also shows and optional hinge 2018 that is used to affix the support 2002 to the removable toilet seat 2012. The hinge 2018 allows the support 2002 to be flipped out of the way if needed, as shown in FIG. 9. Visible in FIG. 8 is a retractable latch 2020 that supports a side of the support 2002 opposite the hinge 2018 when the retractable latch 2020 is in the engaged position, as it is in FIG. 8. In FIG. 9, the retractable latch 2020 is in a disengaged position, so that the support 2002 can be flipped out the way. As can been seen in FIG. 9, the support also includes a slot 2022 and a notch 2024 so that the tubing 16 (not shown) of the enema system 100 (not shown) may be conveniently threaded through the support 2002. FIG. 10 sows a top view of the enema assist device 2000.

In use, a patient in need of the enema assist device 2000 as disclosed herein, i.e. a person having weak muscle tone in the anal sphincter, would place the removable toilet seat 2012 of the enema assist device 2000 on top of a fixed toilet seat on a toilet. The patient would then sit on the removable toilet seat 2012 such that the fluid delivery tip 14 would partially enter their colon, since the fluid delivery tip protrudes beyond the plane of the removable toilet seat. In addition, the support 2002 is sufficiently rigid that it does not appreciably deflect due to the person's weight. Because the fluid delivery tip 14 is tapered, i.e., has a conical shape, and is soft on the outside, but comprises a rigid interior, a water tight seal is formed between the fluid delivery tip 14 and an anal sphincter having weak muscle tone. The enema assist device 2000 thus eliminates the need for a caregiver to reach over and behind the patient to hold the fluid delivery tip in place.

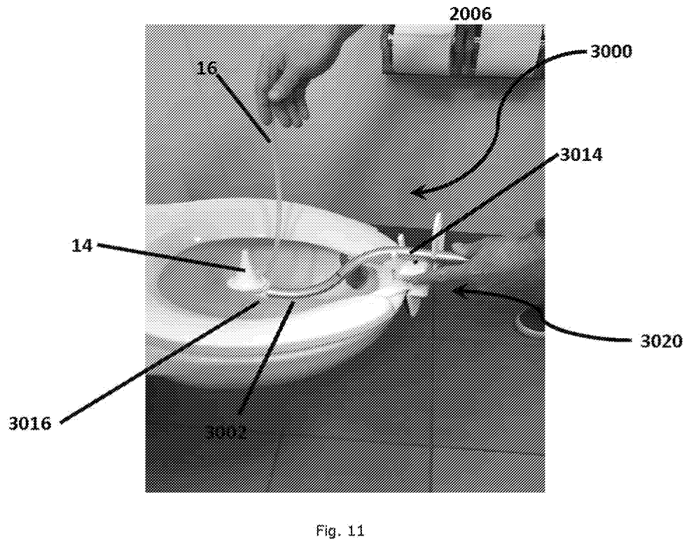

FIGS. 11-13 show a third exemplary embodiment 3000 of the enema assist device. As shown in FIG. 11, this third embodiment 3000 comprises a rigid cantilever 3002 as the support for the fluid delivery tip 14. The rigid cantilever 3002 includes an adjustable attachment system 3020, a first end 3014 and a second end 3016, wherein the first end 3014 is attached to a toilet bowl under the toilet seat with the adjustable attachment system 3020 and the second end 3016 is attached to the fluid delivery tip 14.

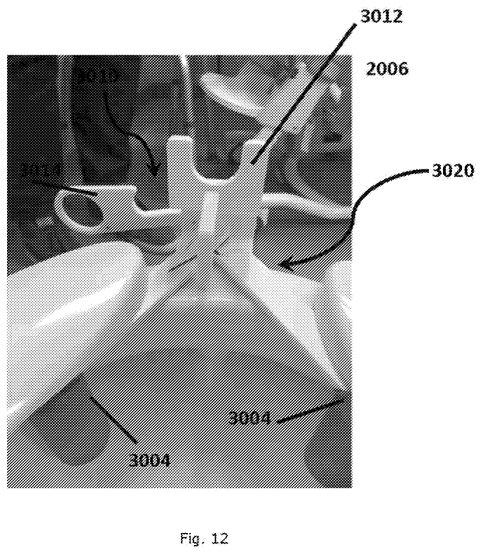

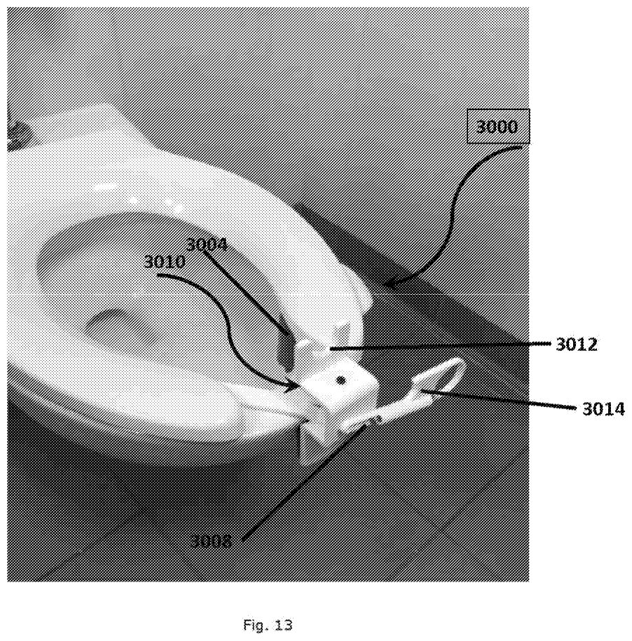

FIGS. 12 and 13 show the adjustable attachment system 3020 that is included in the third embodiment enema assist device 3000 in place on a toilet bowl. The adjustable attachment system 3020 includes at least one clamp 3004 configured to attach to the toilet bowl. As shown in FIGS. 12 and 13, the adjustable attachment system 3020 may include two such clamps 3004. The adjustable attachment system includes at least one adjustment clip 3010 fixedly attached to the clamps 3004. The adjustment clip 3010 has a lower receptacle 3012 configured to hold the first end 3014 (not shown in FIGS. 12 and 13) and an upper arm 3014 pivotally attached to the lower receptacle 3012 and configured to pivot between an open position and a closed position. The adjustment clip also has a spring 3008 (shown in FIG. 13) that is attached to the lower receptacle 3012 and to the upper arm 3014. The adjustment clip 3010 is configured and arranged such that when the upper arm 3014 is in the open position (as shown in FIGS. 12 and 13), the first end 3014 (shown in FIG. 11) can be placed in the lower receptacle 3012 and be moved from front to back relative to the toilet bowl. The adjustment clip 3010 is further configured such that when the upper arm 3014 is in the closed position, the spring 3008 urges the upper arm 3014 against the first end 3014, whereby the first end 3014 is attached to the toilet bowl. As shown in FIG. 11, the rigid cantilever 3002 maybe a curved bar. The rigid cantilever 3002 may be made of metal.

In use, a patient in need of the enema assist device 3000 as disclosed herein, i.e. a person having weak muscle tone in the anal sphincter, would use the clamps 3004 to clamp the enema assist device 3000 to a toilet bowl under a toilet seat. By using the adjustable clip as described above, the enema assist device 3000 may be moved backwards and forwards relative to the toilet seat, depending on the size of the toilet seat and the size of the patient. The patient would then sit on the toilet seat such that the fluid delivery tip 14 would partially enter their colon, since the fluid delivery tip protrudes beyond the plane of the toilet seat. In addition, the rigid cantilever 3002 is sufficiently rigid that it does not appreciably deflect due to the person's weight. Because the fluid delivery tip 14 is tapered, i.e., has a conical shape, and is soft on the outside, but comprises a rigid interior, a water tight seal is formed between the fluid delivery tip 14 and an anal sphincter having weak muscle tone. The enema assist device 3000 thus eliminates the need for a caregiver to reach over and behind the patient to hold the fluid delivery tip in place.

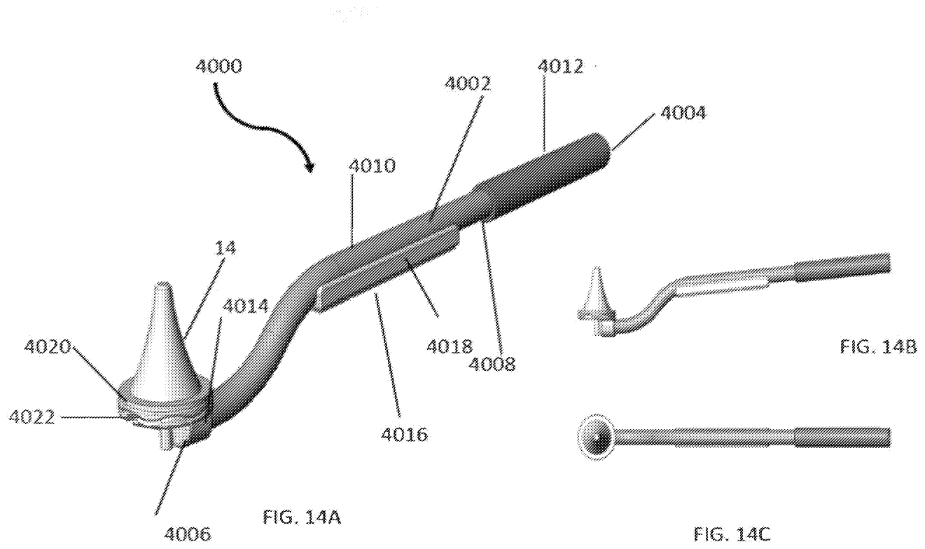

Another embodiment of the invention is shown in FIGS. 14-16. In this embodiment, the support for the enema assist device 4000 is a rigid bar 4002. The rigid bar 4002 may be straight or curved and may be made of a metal or plastic tube for example. This embodiment 4000 also is configured to hold the fluid delivery tip 14 which is configured to be partially insertable into a body orifice of a user and configured to be in fluid communication with an enema delivery system. The rigid bar 4002 has a front 4004, a back 4006, a bottom surface 4008, and a top surface 4010. The rigid bar 4002 is configured to be placed on a toilet bowl (not shown in FIG. 14) such that the front 4004 is oriented towards the front of the toilet bowl.

The rigid bar 4002 is further also configured to support the fluid delivery tip 14 such that the fluid delivery tip is disposed on the top surface of the back of the rigid bar as may be seen in FIG. 14A and in 14B. In use, the fluid delivery tip 14 will protrude at least partially beyond a plane defined by a top of the toilet seat. As may be seen in FIG. 14, the rigid bar includes a hand grip region 4012 located at the front 4004 of the rigid bar 4002 as well as an attachment region 4014 located on the top surface of the back 4006 of the rigid bar 4002. The rigid bar 4002 also includes a pivot region 4016 located on the bottom surface and in between the front 4004 and the back 4006 of the rigid bar 4002. The fluid delivery tip 14 is attached to the attachment region 4014.

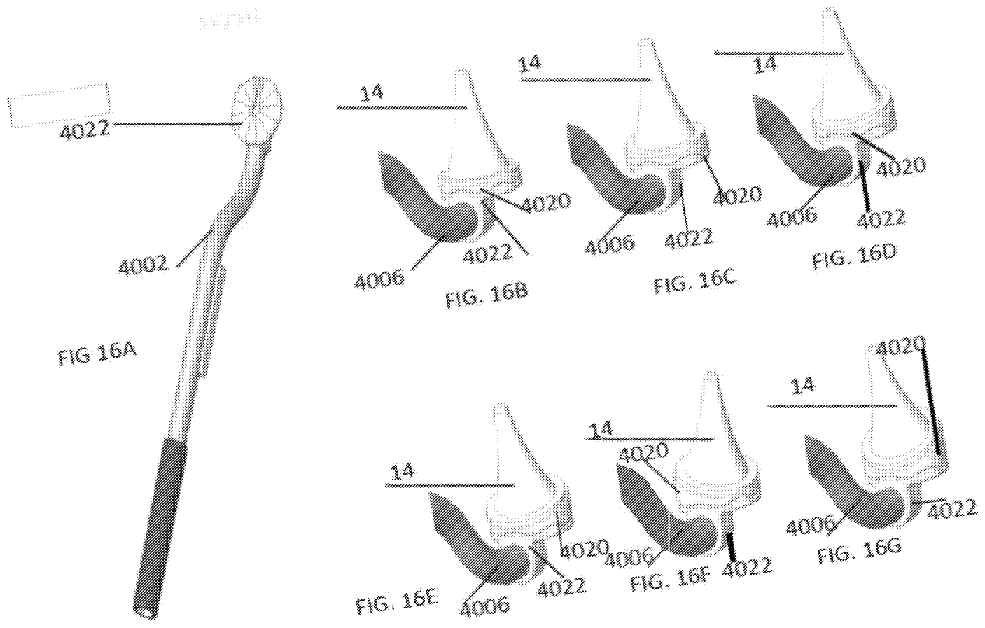

In use, the pivot region 4016 contacts the toilet bowl such that the hand grip region 4012 projects beyond an edge of the toilet bowl and the fluid delivery tip 14 is over a center region of the toilet bowl. A user of the enema assist device 4000 grips the hand grip region 4012 and urges the hand grip region 4012 downward and thereby pivots the fluid delivery tip 14 upward and urges the fluid delivery tip 14 into the body orifice of the user. The hand grip region may be made of a neoprene foam tube 4011 for the patient to comfortably hold the device in place while administering the enema fluid. The pivot region may include a foot 4018 on the bottom of the rigid bar 4002 to help the patient hold the device 4000 in place. The foot 4018 thus contacts and provides a pivot point on the toilet seat or the toilet bowl and allows for adjusting the device 4000 tailored to a patient's need. FIG. 16 shows in an exploded view how the foot 4018 may be attached to the rigid bat 40

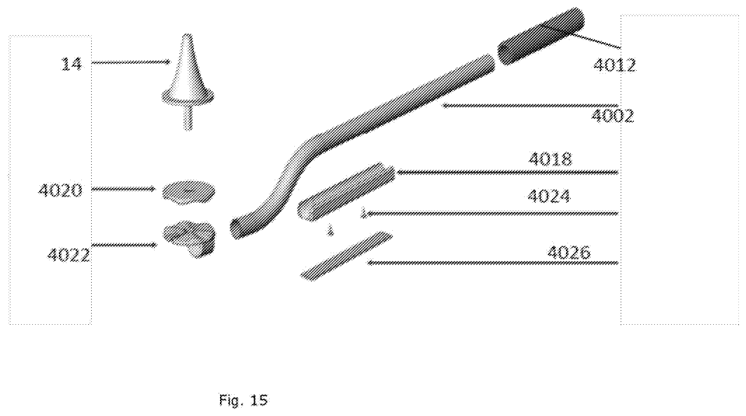

The fluid delivery tip attachment region 4014 may further include at least one angled wedge section 4020 on the top surface thereof and interposed between the fluid delivery tip and the rigid bar, the wedge section 4020 having an angle α, relative to the plane defined by the top of the toilet seat. The wedge section 4020 may also have a rotational angle β, relative to an axis along the length of the rigid bar. In this way, the wedge section 4020 imparts the angle α to the top of the rigid bar and thus angles the fluid delivery tip 14 at the angle α. The wedge section 4020 may be attached directly the rigid bar 4002 or may be attached to a platform 4022 attached to the back end 4006 of the rigid bar 4002. The platform 4022 may have grooves for placement of different angled wedges 4020. The wedges 4020 are designed to fit snuggly in place while allowing for a patient to fit the fluid delivery tip 14 in the appropriate anatomic arrangement. Wedges 4020 can be stacked on one another and may be made from a soft urethane material. The fluid delivery tip 14 fits on top of the platform 4022. FIG. 15 shows these parts in an exploded view. The foot 4018 may be held on the rigid bar 4002 using screws 4024, for example. The screws 4024 may then be covered by an adhesive backed pad 4026.

FIG. 16A shows the rigid bar 4002 and the platform 4022. FIGS. 16B-16G show the fluid delivery tip 14 attached to various wedges 4020 that are attached to the back 4006 of the rigid bar 4002 via the platform 4022. FIGS. 16B-16G show 0° wedge, 5° wedge, 10° wedge, 15° wedge, 15° wedge with 60° rotation, and a 20° wedge, respectively.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made thereto and that the subject matter disclosed herein may be implemented in various forms and examples, and that they may be applied in numerous applications, only some of which have been described herein. It is intended by the following claims to claim any and all modifications and variations that fall within the true scope of the present concepts.

We claim:

1. An enema assist device comprising:
a support, wherein the support has a front, a back, a bottom surface, and a top surface, wherein the support is configured to be placed on a toilet bowl such that the support front is oriented towards the front of the toilet bowl,
the support further being configured to support a fluid delivery tip such that the fluid delivery tip is disposed on the top surface of the support and protrudes at least partially beyond a plane defined by a top of the toilet seat.

2. The enema assist device according to claim 1, wherein the top surface of the support slopes at an angle α, relative to the plane defined by the top of the toilet seat, thereby angling the fluid delivery tip at the angle α.

3. The enema assist device of claim 1, wherein the support further comprises at least one angled wedge section on the top surface thereof and interposed between the fluid delivery tip and the support, the wedge having an angle α, relative to the plane defined by the top of the toilet seat, thereby imparting the angle α to the top of the support and thus angling the fluid delivery tip at the angle α.

4. The enema assist device according to claim 3, wherein the top surface of the support slopes downward from back to front at an acute angle α, thereby positioning the fluid delivery tip at an angle toward the toilet front.

5. The enema assist device according to claim 1, wherein the support comprises a platform, the platform comprising a first side and an opposing second side, wherein the first side and the second side of the platform are placed under opposing sides of the toilet seat, and the support front is oriented towards the toilet front.

6. The enema assist device according to claim 1, wherein the toilet seat comprises a removable toilet seat configured to be placed on top of a fixed toilet seat on a toilet bowl.

7. The enema assist device according to claim 1 wherein the support comprises a rigid cantilever, wherein the rigid cantilever comprises an adjustable attachment system, a first end and a second end, wherein the first end is attached to a toilet bowl under the toilet seat with the adjustable attachment system and the second end is attached to the fluid delivery tip.

8. The enema assist device according to claim 7, wherein the adjustable attachment system comprises:
at least one clamp configured to attach to the toilet bowl,
an adjustment clip fixedly attached to the at least one clamp, the adjustment clip comprising:
a lower receptacle configured to hold the first end,
an upper arm pivotally attached to the lower receptacle and configured to pivot between an open position and a closed position, and
a spring attached to the lower receptacle and to the upper arm,
wherein when the upper arm is in the open position, the first end can be placed in the lower receptacle and be moved from front to back relative to the toilet bowl, and wherein when the upper arm is in the closed position, the spring urges the upper arm against the first end, whereby the first end is attached to the toilet bowl.

9. The enema assist device according to claim 7, wherein the rigid cantilever comprises a curved bar.

10. The enema assist device according to claim 9, wherein the second end further comprises at least one angled wedge section on the top surface thereof and interposed between the fluid delivery tip and the second end, the wedge having an angle α, relative to the plane defined by the top of the toilet seat, thereby imparting the angle α to the top of the top surface of the second end and thus angling the fluid delivery tip at the angle α.

11. The enema assist device according to claim 10, wherein the at least one angled wedge section further has a rotational angle β measured relative to an axis along a length of the rigid cantilever.

12. The enema assist device according to claim 1, wherein the support comprises a rigid bar, the rigid bar comprising a hand grip region located at the front of the rigid bar, an attachment region located on the top surface of the back of the rigid bar, and a pivot region located on the bottom surface and in between the front and the back of the rigid bar, the fluid delivery tip being attached to the attachment region and, wherein
in use, the pivot region contacts the toilet bowl such that the hand grip region projects beyond an edge of the toilet bowl, the fluid delivery tip is over a center region of the toilet bowl, and a user of the enema assist device grips the hand grip region and urges the hand grip region downward and thereby pivots the fluid delivery tip upward and urges the fluid delivery tip into the body orifice of the user.

13. The enema assist device according to claim 12, wherein the fluid delivery tip attachment region further comprises at least one angled wedge section on the top surface thereof and interposed between the fluid delivery tip and the support, the wedge having an angle α, relative to the plane defined by the top of the toilet seat, thereby imparting the angle α to the top of the support and thus angling the fluid delivery tip at the angle α.

14. The enema assist device according to claim 12, wherein the at least one angled wedge section further has a rotational angle β measured relative to an axis along a length of the rigid bar.
